(19) United States
(12) Patent Application Publication
(10) Pub. No.: US 2009/0123564 A1
(43) Pub. Date: May 14, 2009

(54) NOVEL COMPOSITIONS FOR HAIR DISORDERS AND PROCESS OF PREPARATION THEREOF

(76) Inventors: Rajesh Jain, New Delhi (IN); Kour Chand Jindal, New Delhi (IN); Aniruddha Datta, New Delhi (IN)

Correspondence Address: LADAS & PARRY LLP, 26 WEST 61ST STREET, NEW YORK, NY, 10023, US

(21) Appl. No.: 12/294,542

(22) PCT Filed: Mar. 19, 2007
(86) PCT No.: PCT/IN07/00111

§ 371 Date: Sep. 25, 2008

(30) Foreign Application Priority Data
Mar. 31, 2006 (IN) 930/DEL/2006

(51) Int. Cl. A61K 33/30; A61K 36/00; A61K 36/886; A61P 17/14; A61K 36/16; A61K 36/61

(52) U.S. Cl. 424/642; 424/725; 424/744; 424/752; 424/742

(57) ABSTRACT

Novel compositions for hair loss prevention and/or hair growth promotion comprising at least active agent preferably derived from natural source such as from the plant Vernonia sp., either alone or in combination with other active agent(s) and optionally one or more excipient(s) are provided. The process for the extraction of hair growth promoting agent and preparation of compositions comprising such active agent are also described. The novel composition is preferably in the form of an oral or topical preparation such as tablet, capsule, liquid solution or suspension, cream, gel, lotion or spray and is useful against hair disease(s)/disorder(s) and/or other associated disorders particularly in the management of testosterone induced androgenic alopecia.

### FIELD OF THE INVENTION

The present invention relates to novel compositions for hair loss prevention and/or hair growth promotion comprising at least one agent preferably derived from natural source as active agent, either alone or in combination with other active agent(s), and at least one carrier, optionally with one or more other excipient(s). The active agent is preferably extracted from the plant Vernonia sp. The present invention also describes process for extraction of the hair growth promoting agent and also process of preparation of compositions comprising such active agent. Also the present invention provides method of using such compositions. The novel composition is in the form of an oral preparation such as tablet or capsule, or a topical preparation such as liquid solution or suspension, cream, gel, lotion or spray. The compositions are particularly useful against hair disorders and/or other associated disorders particularly in the management of testosterone induced androgenic alopecia. The compositions of the present invention are useful as a pharmaceutical or a cosmetic or an ayurvedic product.

### BACKGROUND OF THE INVENTION

The use of herbal products for medicinal benefits has played an important role in nearly every culture on earth. Herbal medicine was practiced by ancient people in Africa, Asia, Europe, and the Americas. The recent increase in the use of herbal products is associated with the belief that herbs can provide some benefits over, and above allopathic medicine and allows users to feel that they have some control in their choices of medications. In India particularly, Ayurveda is an example of a long standing tradition that offers a unique insight into approaches for the prevention and/or treatment of various human ailments. These herbal products are safe compared to allopathic drugs.

Vernonia anthelmintica plant is cultivated by the Sri Lankans, and is in great repute as a remedy, which is indicated by its name. The bitter, nauseous, black seeds of this plant, in doses of 50 to 60 grains, are valued in Sri Lanka as an anthelmintic and are commonly used for expelling the Ascaris lumbricoides, and also as a vermicide. The dose of the powdered seed to an adult is from ½ to 1 drachm. The native physicians prescribe it generally as a tonic in the shape of an infusion. The Sri Lankan name and the Tamil name of Vernonia anthelmintica is sanne nayan and kado-seragam respectively. The Vernonia plant is a small herb found all over India and its powdered seed is especially mixed with honey to expel intestinal worms, cough and indigestion. It is also commonly referred to as Iron weed and is a very common plant in the Western states, growing in the woods and prairies, and along river streams, and flowering from July to September. The root, which is the part used, is bitter, and imparts its properties to water or alcohol. Iron weed is a bitter tonic, deobstruent, and alternative. In powder or decoction, the root is beneficial in amenorrhoea, dysmenorrhoea, leucorrhoea, and menorrhagia. In intermittent, remittent and bilious fevers, the decoction or a saturated tincture has been recommended. It is also said to have been useful in scrofula, and some cutaneous diseases. Dose of the decoction is usually 1 to 2 fluid ounces; of the tincture, 1 to 2 fluid drachms. The leaves or powdered root in the form of poultice make an excellent discutient application to tumors. Several species of the plant Vernonia such as Vernonia noveboracense, Willdenow, and its variety, V. praealta, bearing purple flowers, and the V. tomentosa, with some other species possess similar medicinal properties as Vernonia anthelmintica. The root of V nigritiana, Oliver, of West Africa, is used in Senegambia, under the name of batiatior, as a febrifuge. It contains the glucoside namely vernonin (Heckel and Schlagdenhauffen, Amer. Jour. Pharm., 1889, p. 40).

Vernonia belongs to the Asteraceae family. Its sesquiterpene lactones have demonstrated anti-tumor activity, and the Vernonia chemicals (vernoniosides) of the pith have proven effective against drug-resistant malarial parasites, which are very common within the range of this plant. Vernonia have been part of Tanzanian folk medicine for hundreds of years. The WaTongwe traditionally use Vernonia for stomachaches and several parasitic infections. V. latifolia has been reported to stop bleeding by inducing clot formation. The leaves are used in soup and stew as a strength-giving tonic by the local people after soaking them in water and cooking them. They also widely use Vernonia to treat parasites and other ailments in themselves and their livestock, indicating potential agricultural applications for other countries. Additionally, it is documented that Vernonia is used locally as an insecticide.

Study has been conducted to evaluate the effect(s) of a novel water-soluble leaf extract of Vernonia amygdalina (VA) on human breast cancer cell DNA synthesis. MCF-7 cell line, considered a suitable model, was used in this study. Treatment of cells with physiologically relevant concentrations of water-soluble VA extract potently inhibited DNA synthesis in a concentration-dependent fashion both in the absence and presence of serum. The studies demonstrate anticancer activities of both crude and fractions of a water-soluble leaf extract of VA. Earlier investigators have shown that purified fractions of chloroform extract of VA elicited anticancer effects in human carcinoma of the nasopharynx. The process began with chloroform extraction of VA dried leaves to generate fractions A and B. Purification of A between 10% aqueous methanol and petroleum ether yielded an aqueous methanol fraction D. Fractionation of fraction D with silicic acid chromatography resulted in two cytotoxic fractions called F and H. Further chromatography of fraction H produced colorless oil called vemodaline, while rechromatography of the cytotoxic fraction F yielded two similar crystalline compounds, vernolide and vernomygdine. These three pure fractions elicited cytotoxic effects in human carcinoma nasopharynx cells with IC₅₀ values of 1.8, 2.0, and 1.5 μg/ml respectively. The investigators concluded that the activities of these three compounds were dependent on their possessions of the α-methyl-γ-lactone group as part of their structures. Jisaka and colleagues also showed that vemodaline and vernolide elicited antitumoral effects in leukenia cells P-388 and L-1210 with IC₅₀ values of 0.11 and 0.17 μg/ml for vernodaline and 0.13 and 0.11 μg/ml for vernolide, respectively.

Phytochemical analysis of V. amygdalina samples collected at Mahale from individual plants known to be used by chimpanzees revealed the presence of two major classes of bioactive compounds. A number of known sesquiterpene lactones, and 13 new stigmastane-type steroid glucosides and their freely occurring aglycones, have been isolated (Ohigashi et al. 1991, Jisaka et al. 1992a, 1992b, 1993a, 1993b). The sesquiterpene lactones present in V. amygdalina are also found in V. colorata and in a number of other Vernonia species. They are well known for their anthelmintic, antiamoebic, antitumor, and antibiotic properties (Toubiana and Gaudemer 1967, Kupchan et al. 1969, Asaka et al. 1977, Gasquet et al. 1985, Jisaka et al. 1992a, 1993b). Crude methanol extracts of the leaves exhibited immunosuppressive activity and inhibition of the process that initiates the first stage of tumor cell growth (Koshimizu et al. 1993). The cytotoxic sesquiterpene lactones were found to be most abundant in the leaves and bark. Vernonia leaves are thus used to treat stomachaches and parasitic infections, and the plant is also used as an insecticide, strength giver, and a blood dotter (McGraw Hill, 2000). Further, a traditional report from Indian traditional medicine has found that the tropical plant Vernonia antheltmintica seeds are both anti-inflammatory and cytotoxic.

Alopecia is the absence or slowing of hair growth in an area of the body where hair formerly grew. It may be caused by physical damage to the hair itself or to the hair follicles, but it is most often the result of changes in the natural growth cycle of hair. In some types of alopecia, the growth cycle is disrupted by some temporary situation such as a chemical imbalance or stress. However, the vast majority (95%) of cases of hair loss in both men (male pattern baldness) and women (female diffuse baldness) are genetic in origin. Below the surface of the skin is the hair root, which is enclosed within a hair follicle. At the base of the hair follicle is the dermal papilla (or papilla). The dermal papilla is fed by the bloodstream which carries nourishment to produce new hair. The dermal papilla is a structure very important to hair growth because it contains receptors for male hormones and androgens. Androgens regulate hair growth and in scalp hair androgens may cause the hair follicle to get progressively smaller and contribute to the development of alopecia (Hoffmann, 2001).

Causes of hair loss are varied. Six major types of hair loss are namely alopecia greata, androgenetic alopecia, anagen effluvium (cancer treatment hair loss), self induced hair loss, telogen effluvium and scarring alopecia. Other types of hair loss include syphilitic alopecia (usually a manifestation or secondary syphilis), scleroderma (a disease that causes fibrosis i.e. hardening and tightening of the skin which interferes with the normal functioning of the hair follicles and growth of the hair and tinea capitis (which causes hair loss by digesting the keratin of the hair). Alopecia greata is thought to be an auto-immune disease of the hair, initially appearing as a rounded bare patch about an inch across the sclap. Alopecia greata affects both men and women equally and is often experienced first in childhood. Androgenetic alopecia accounts for 95% of all hair loss. It can affect both men and women although men experience a much greater degree of loss. When androgenetic alopecia occurs, large active hair follicles in specific areas begin to change to smaller less active ones that shrink slightly with each new growth cycle. Testosterone 5α-reductase converts testosterone to 5α-DHT. 5α-DHT causes the hair shafts to narrow producing progressively finer hairs with each new growth cycle until eventually the hair becomes transparent and stop emerging. Thus this male hormone, 5α-DHT contributes to androgenetic alopecia in those who are genetically predisposed. It is interesting to note that individuals with a deficiency in testosterone 5α-reductase do not develop androgenetic alopecia. This is because the body is unable to convert testosterone into 5α-DHT (Hoffmann et al., 2000: Hibberts et al., 1998). Anagen Effluvium is the sudden hair loss which occurs as a result of chemicals or radiation, such as the hair loss that results during certain types of chemotherapy or radiation treatment. The hair loss is usually sudden occurring 1 to 3 weeks after expose to the chemicals or radiation has occurred. Damage to the hair in some cases is self inflicted sometimes consciously or unconsciously. The two main types of self induced hair loss are trichotillomania (which results from the continuous pulling or plucking of the hair) and traction alopecia (usually caused by continuous and excessive pulling on the hair due to various types of hairstyling). Telogen Effluvium occurs when sudden or severe stress causes an increase in the shedding of hair (Brajac et al., 2003). Scarring alopecia occurs when there is inflammation in the hair follicles due to infection. It is easy to identify a case of severe scarring alopecia because there will be rough patches on the surface of the scalp made up of small blood vessels and connective tissue. Scarring alopecia is mainly caused by Discoid Lupus Erythematosus, Lichen Planus, Pseudopelade of Brocq, Aplasia Cutis Congentia or Congenital Atrichia

Several drugs are available to treat alopecia such as 5α-DHT inhibitors (Anti-androgens), SOD (Super oxide dismutases) mimetics, Vasodilators, Activation of PGHS-1 (Prostaglandin-H synthase-1) and Potassium channel openers (PCOs).

The main cause of hair loss is the binding of 5α-DHT to the androgen receptors. The term “5α-DHT inhibitor” is used for the substances that inhibit enzymes responsible for producing 5α-DHT, such as testosterone 5α-reductase, or otherwise block or mask activity of 5α-DHT by binding to 5α-DHT thereby inactivating it and/or binding to 5α-DHT receptors. The 5α-DHT inhibitors used in the treatment of alopecia are saw palmetto extract, nettle root extract, azelaic acid and Ginkgo biloba (Hiipakka et al., 2002: Kaufman, 2002). Super oxide dismutases are enzymes which destroy super oxide free radical, an important biological mediator. In many systems super oxide opposes the action of another ubiquitous messenger substance, nitric oxide. Nitric oxide is natural hair-growth stimulating factor. SOD mimetics include prazotide copper and a copper-binding peptide. TEMPOL (4-Hydroxy-2,2,6,6-tetramethyl piperidinyl oxyl or 4-Hydroxy TEMPO) is another SOD mimetic. Vasodilators act by increasing the amount of blood to the hair follicle. Topical minoxidil was originally a drug for hypertension and because it is a vasodilator, it is now the most widely recommended treatment for androgenetic alopecia. Recent studies have shown that topical minoxidil does not cause an increase in skin blood flow which was originally thought to be the mechanism by which minoxidil works. Studies carried out suggest that minoxidil works by activating PGHS-1 which helps in promoting hair growth (Messenger et al., 2004). The opening of intracellular potassium channels is a common mechanism of action for a set of anti-hypertensive drugs that includes the hair-growth-inducing agent minoxidil. Recent work suggests PCOs also influence hair growth.

Treatment of alopecia greata involves use of corticosteroids (topical application), dithranol, retin A (tretinoin), topical minoxidil (marketed as Regaine, Rogaine or Headway) and zinc (Alabdulkareen et al., 1998: Meidan et al., 2001) or administration of systemic cortisone, PUVA treatment, or use of irritants. Treatments for androgenetic alopecia particularly for male pattern baldness include minoxidil (most widely used), propecia (finasteride), retin-A (tretinoin), zinc and skinoren/azelaic acid (Kaufman et al., 1999) and for female pattern baldness include diane 35 (cyproterone acetate with ethinyloestradiol), cimetidine, cyproterone acetate, spironolactone, nizoral/ketoconazole. The treatment for trichotillomania often involves counseling or psychiatric help; however in some cases an antidepressant may be prescribed. Generally a change in hairstyle that reduces the traction on the hair and hair follicle is all that is required in the treatment of traction alopecia. Telogen Effluvium which is the hair loss caused by child birth, pregnancy termination, starting or stopping birth control pills, drug therapy, severe emotional stress, etc. are usually temporary an in most cases hair will grow back normally soon after it has fallen out. Treatment of scarring alopecia includes use of topical corticosteroid ointments such as triamcinolone acetonide, anti malarial drugs such as hydroxychloroquine, steroid lotions, etc. For syphilitic alopecia, penicillin is often used to treat the condition. Tinea Capitis, which is the hair loss caused by ringworm infection, involves use of commonly used treatment for ringworm such as an anti fungal agent e.g. Nizoral shampoo (ketaconazole 2%). Natural Products in the treatment of alopecia include Aloe (Aloe barbadensis), Burdock (Arctium minus), Capsicum (Capsicum annuum L), Ginger (Zingiber officinale), Ginkgo (Ginkgo biloba), Green Tea (Camellia sinesis), Hip (Rosa canina), Lavender (Lavendula officinale), Milfoil (Achillea millefolium), Onion (Allium cepa), Pygeum (Pygeum africanum), Rattanjot (Arnebia sp.), Red Pepper (Capiscum annum), Rosemary (Rosamarinus officinalis), Safflower oil (Carthamus tinctorious), Saw Palmetto (Serenoa repens), Stinging Nettle (Urtica dioica) and Tea Tree Oil (Melaleuca alternifolia).

Aloe barbadensis contains an enzyme called superoxide dismutase and activates the production of nitric oxide that stimulates hair re-growth in those suffering from male pattern baldness. Emollient properties also protect against damage to the scalp and hair. Arctium minus extract helps reverse scalp conditions due to alopecia and promotes recovery of scalp irritation. It also helps to improve hair strength, shine and body with its natural phytosterols and essential fatty acids. Capsicuni annuum L stimulates hair growth by 50% and increases blood flow to the scalp as well as histamine release to stimulate cell division. It is excellent at accelerating new hair growth. Ginger has circulatory agents that stimulate the hair follicle's growth cycle. Additionally, ginger is rich in fatty acids which are recommended for hair loss, and the thinning of the hair shaft. Ginkgo extracts have 5α-DHT inhibitory activity and hence possess hair growth stimulatory activity. It also enhances the microcirculation in the roots and hence stimulates hair growth. It is thought that natural chemicals called catechins found in green tea may inhibit the enzyme type-I testosterone 5α-reductase which converts testosterone into 5α-DHT. Green tea is therefore believed to effective in preventing and treating male pattern type baldness. Hip extract is rich in vitamins C, B1, B2, pyrophosphate P, K and E, tannins, pectin and fruit acids. Vitamin B1 takes part in skin moisture balance and is essential for normal skin functions. Vitamin B2 decreases sebaceous secretions and prevents hair loss. It improves local circulation, thus stimulating hair growth and nourishing skin. Lavender has very strong anti-inflammatory properties to help combat alopecia. It provides shine, volume and lift without striping hair color. Achillea millefolium extract contains essential oils, tannins and organic acids. The extract has healing, anti-inflammatory and soothing action and stimulates hair growth. Nettles have been used to treat alopecia due to their effectiveness in blocking 5α-DHT. Allium cepa extract has a high sulfur content which is believed to be a hair-healing mineral. Pygeum africanum extract inhibits the enzyme type-I testosterone 5α-reductase. It is widely used to prevent male pattern baldness. Ainebia sp. root extract of the plant yields a napthaquinone named as shikonin. Shikonin is testosterone 5α-reductase inhibitor and so is proposed to have hair fall preventive activity. Capiscum annum extract acts as a skin irritant to draw blood and nutrients to the scalp and also encouraged the release of histamines that stimulated cell division and hair re-growth. Rosemary has been shown to promote increased circulation as well as help remove dandruff and sebum accumulations on the scalp. Carthamus tinctorious extract acts as a vasodilator that dilates blood vessels. This allows more blood to deliver nutrients to the hair follicles. Serenoa repens extract is very effective at blocking the formation of 5α-DHT and appears to block the androgen receptors which are found on the hair follicles. It blocks both the type-1 and type-2 forms of testosterone 5α-reductase. Urtica dioica extract is thought to block the conversion of testosterone into 5α-DHT. The essential oil extract of Melaleuca alternifolia has shown to kill the yeast (Pityrosporum ovale) responsible for causing dandruff. This yeast infects the scalp, causing inflammation and itching leading to hair loss. Tea tree oil soaks through the skin and kills the yeast so that the hair is free from dandruff and hence reduces the hair loss. The bioactive constituents such as essential oils derived from pine needles and burdock roots; tannins (oak and willow bark); bioflavonoids (willow bark, pine needles and rice husks) and vitamins B1, B6 and B7 (rice husks and pine needles) have shown to possess hair growth promoting activity.

It is well known in the art that certain undesirable physiological manifestations, such as acne vulgaris, seborrhea, female hirsutism, male pattern baldness and benign prostatic hypertrophy, are the result of hyperandrogenic stimulation caused by an excessive accumulation of testosterone or similar androgenic hormones in the metabolic system. Hair growth depends on a close interaction of different cell populations of the hair follicle. In certain regions of the body, androgens interfere with this highly regulated cooperation in a yet poorly understood manner. The response of hair follicles to androgens can be categorized as androgen-dependent, e.g. in the beard, androgen-sensitive, e.g. in the frontal scalp of affected individuals, or androgen-independent, e.g. in the occipital scalp. At the target cell level, the balance between 5 alpha-reductase, 17 beta-hydroxysteroid-dehydrogenase (17 beta-HSD) and 3 alpha-hydroxysteroid dehydrogenase (3 alpha-HSD) yields metabolites with different androgenic potential. Early attempts to provide a chemotherapeutic agent to counter the undesirable results of hyperandrogenicity resulted in the discovery of several steroidal antiandrogens having undesirable hormonal activities of their own. The estrogens, for example, not only counteract the effect of the androgens but have a feminizing effect as well. Non-steroidal antiandrogens have also been developed, for example, 4′-nitro-3′-trifluoromethylisobutyranilide. However, these products, though devoid of hormonal effects, are peripherally active, competing with the natural androgens for receptor sites, and hence have a tendency to feminize a male host or the male fetus of a female host. It recently became known in the art that the principal mediator of androgenic activity in some target organs is 5 alpha-dihydrotestosterone, and that it is formed locally in the target organ by the action of testosterone-5 alpha-reductase. It therefore has been postulated and demonstrated that inhibitors of testosterone-5 alpha-reductase will serve to prevent or lessen symptoms of hyperandrogenic stimulation.

Finasteride (Propecia®) is a specific type II 5-alpha reductase inhibitor. That is, it inhibits the enzyme responsible for regulating conversion of testosterone to dihydrotestosterone (DHT). By reducing DHT levels in the scalp, the drug decreases DHT's effects on the hair follicles, reversing the process of hair loss. Finasteride inhibits expression of the enzyme, 5-alpha reductase, which regulates production of dihydrotestosterone (DHT). By lowering DHT levels in the scalp, it reduces DHT's harmful effect on hair follicles. Finasteride decreases DHT concentrations in the serum and the scalp by up to 70% and 60%, respectively.

Recently, there has been increased interest in the use of natural therapy for treatment of a diseased state. A number of herbal extracts have been demonstrated to be useful in the treatment of benign prostatic hyperplasia. One such herbal extract is the extract of the berries of Saw Palmetto. Saw Palmetto is a small palm tree with large leaves and large deep red black berries. Saw Palmetto berries contain an oil with a variety of fatty acids and phytosterols. The fat soluble extract of Saw Palmetto berries has been shown to inhibit the conversion of testosterone, which is thought to be responsible for the enlargement of the prostrate. In addition, Saw Palmetto extract inhibits the binding of DHT to receptors, thus blocking DHTs action and promoting the breakdown of the potent compound. Another herbal extract utilized is African Pygeum. Pygeum is a large evergreen tree growing in the higher plateaus of southern Africa. The bark of the tree is processed to produce a fat-soluble fraction, which contains phytosterols, pentacyclic triterpenoids and ferulics esters of long chain fatty acids. African Pygeum extracts in double blind clinical trials have been found to be effective in treating a wide range of prostatic hyperplasia. Consumption of Pygeum extract resulted in a significant amelioration of symptoms, reduction in prostate size and clearance of bladder neck urethral obstruction. Stinging nettles extract, which are an extract of a perennial plant growing worldwide, have been demonstrated to show a reduction in prostatic growth potential in mice with the administration of a high dosage of the nettle root extract Stinging nettles have also been traditionally been known as a hair and skin tonic, stimulating hair growth, improving condition of the hair and skin and treating dandruff. There still remains a need for a natural hair growth stimulant for use in treating androgenetic alopecia, having reduced side effects and risk of toxicity compared with synthesized pharmaceutical compounds. The conversion of testosterone (T) to dihydrotestosterone (DHT) via the enzyme 5-alpha reductase (5AR) contributes to hair loss.

No literature has been found relating to the use of Vernonia sp. extract for the treatment of hair loss. There still exists a need for a continued search for novel natural products that may be used for prevention of hair loss and/or regeneration of hair devoid of any toxic effects and preferably which aids in restoration of hair. The present invention not only provides a novel solution to the aforementioned problem but also describes pharmaceutical compositions comprising an extract of a natural product highly effective for treatment of such hair related disorders.

### SUMMARY OF THE INVENTION

It is an objective of the present invention to provide novel compositions for hair loss prevention and/or hair growth promotion comprising at least one agent(s) derived from a natural source or synthetic source or semi-synthetic source as the active agent, either alone or in combination with other active agent(s) and optionally one or more excipient(s).

It is an objective of the present invention to provide novel compositions for hair loss prevention and/or hair growth promotion comprising an extract obtained from the plant Vernonia sp. as the active agent, either alone or in combination with other active agent(s) and optionally one or more excipient(s).

It is an objective of the present invention to provide novel compositions for hair loss prevention and/or hair growth promotion comprising an extract obtained from the plant Vernonia anthelmintica as the active agent, either alone or in combination with other active agent(s) and optionally with one or more excipient(s).

It is another objective of the present invention to provide process for preparation or extraction of the hair loss preventing and/or hair growth promoting agent from a natural source or synthetic source or semi-synthetic source.

It is another objective of the present invention to provide process for preparation of compositions comprising the hair loss preventing and/or hair growth promoting active agent.

It is a further objective of the present invention to provide process for the preparation of such novel composition which comprises the following steps:
i) mixing the hair loss preventing and/or hair growth promoting active agent(s) with one or more excipient(s), and
ii) formulating the mixture into a suitable dosage form.

It is a further objective of the present invention to provide a method of using such novel hair loss preventing and/or hair growth promoting agent or pharmaceutical compositions comprising such agent which comprises administering to a subject in need thereof an effective amount of such agent or composition thereof.

The novel compositions of the present invention are preferably in the form of oral or topical preparations, more preferably in the form of topical preparations such as liquid, cream, gel, lotion or spray. The compositions are useful for hair loss prevention and/or hair growth promotion preferably for the treatment of testosterone induced androgenic alopecia.

### DETAILED DESCRIPTION OF THE INVENTION

The present invention provides novel compositions for hair loss prevention and/or hair growth promotion comprising at least one agent(s) derived from a natural source or synthetic source or semi-synthetic source as the active agent, either alone or in combination with other active agent(s) and optionally one or more excipient(s).

Preferably the hair loss preventing and/or hair growth promoting agent is derived from a natural source. In an embodiment, the composition of the present invention is useful as a pharmaceutical or a cosmetic or ayurvedic product. In an embodiment of the present invention, the novel compositions for hair loss prevention and/or hair growth promotion comprises an extract obtained from the plant Vernonia sp. as the active agent, either alone or in combination with other active agent(s) and optionally one or more excipient(s). In a preferred embodiment of the present invention, the novel compositions for hair loss prevention and/or hair growth promotion comprises an extract obtained from the plant Vernonia anthelmintica as the active agent, either alone or in combination with other active agent(s) and optionally one or more excipient(s). In another embodiment, the plant part used for preparing the extract may be either any part or mixture of parts or whole plant. The parts used are preferably selected from aerial parts such as leaves, flowering tops, flowers, seeds, fruits and stems, or combination of such parts.

In another embodiment of the present invention is provided a process for preparation or extraction of the hair loss preventing and/or hair growth promoting agent from a natural source or synthetic source or semi-synthetic source, or a combination of such sources.

In an embodiment of the present invention, the process of extraction of the hair loss preventing and/or hair growth promoting agent comprises the following steps:
i) Extraction of dried and powdered plant or part(s) of plant with a non-polar solvent or mixtures thereof,
ii) Distillation of the extract to remove the solvent,
iii) Optionally, further extraction of the residue with a polar solvent or mixtures thereof,
iv) Optionally, distillation of the extract to remove the solvent to obtain the desired extract preferably as a powder.

In another embodiment, the process for extraction of the hair loss preventing and/or hair growth promoting agents from Vernonia species comprises the following steps:
i) Extraction of dried and powdered plant or part(s) of plant with a polar solvent or mixture thereof,
ii) Optionally, distillation/concentration of the extract to remove/reduce the solvent,
iii) Optionally drying the extract to remove the solvent to obtain the desired extract preferably as a powder.

In another embodiment, the process for extraction of the hair loss preventing and/or hair growth promoting agent(s) from Vernonia species comprises the following steps:
i) Expression of the juice of the fresh plant or part(s) of plant optionally with addition of a polar solvent or mixture thereof,
ii) Filtration of the juice,
iii) Optionally, distillation/concentration of the extract to remove/reduce the solvent,
iv) Optionally drying the extract to remove the solvent to obtain the desired extract preferably as a powder.

The polar solvent useful in the present invention is selected from but not limited to acetone, methanol, ethanol, isopropyl alcohol such as isopropanol, butanol, water, and the like used either alone or in combination thereof. The non-polar solvent useful in the present invention is selected from but not limited to pentane, hexane, heptane, diethyl ether, petroleum ether, chloroform, dichloromethane, dichloroethane, or mixtures thereof. The mode of drying employed in the invention is selected from a method known to art including but not limited to tray drying, vacuum tray drying, agitated vacuum tray drying, spray drying, freeze drying, lyophilization and the like used either alone or in combination thereof.

In a further embodiment of the present invention is provided a process for the preparation of novel composition comprising the hair loss preventing and/or hair growth promoting agent, which comprises the following steps:
i) mixing the hair loss preventing and/or hair growth promoting active agent(s) with one or more excipient(s), and
ii) formulating the mixture into a suitable dosage form.

In an embodiment, the hair loss preventing and/or hair growth promoting agent comprises one or more phytosterol(s). The phytosterol(s) are either extracted from the natural source such as those obtained from Vernonia sp. or synthesized by using a combination of the synthetic techniques known to the art. The phytosterol(s) may alternatively be obtained semi-synthetically. In an embodiment, the extract for hair loss prevention and/or hair growth promotion also comprises one or more components such as fatty acids or fatty acid esters, carotenoids, and the like or mixtures thereof. In an embodiment, the Vernonia extract may be subjected to column chromatography for isolation of the phytochemical constituent(s). Veronia anthelmintica extract may be column chromatographed using alumina neutral as stationary phase and 15% chloroform in hexane as mobile phase. Fractions obtained may be pooled and dried. Pooled fraction may again be column chromatographed using silica gel (100-200) as stationary phase and hexane as mobile phase. Polarity of mobile phase may be increased to 7% chloroform in hexane. The fractions obtained in this mobile phase may be pooled and dried under vacuum; and the pooled fraction may be crystallized using a suitable solvent to obtain a pure compound.

In an embodiment, the extract for hair loss prevention and/or hair growth promotion comprises one or more extract(s) obtained from several species of the plant Vemonia such as but not limited to Vernonia noveboracense, Vernonia praealta, Vernonia tomentosa, Vernonia anthelmintica, Vernonia amygdalina, Vernonia cinerea, and the like.

In an embodiment, the novel extract of the present invention obtained from the plant Vernonia sp. is combined with at least one other extract obtained from a natural source including but not limited to a group comprising Aloe (Aloe barbadensis), Burdock (Arctium minus), Capsicum (Capsicum annuum L), Ginger (Zingiber officinale), Ginkgo (Ginkgo biloba), Green Tea (Camellia sinesis), Hip (Rosa canina), Lavender (Lavendula officinale), Milfoil (Achillea millefolium), Nettles (Urtica dioica), Onion (Allium cepa), Pygeum (Pygeum africanum), Rattanjot (Arnebia sp.), Red Pepper (Capiscum annun), Rosemary (Rosamarinus officinalis), Safflower Oil (Carthamus tinctorious), Saw Palmetto (Serenoa repens), and Tea Tree Oil (Metaleuca alternifolia).

In a further embodiment of the present invention is provided a method of using the novel hair loss preventing and/or hair growth promoting agent or pharmaceutical compositions comprising such agent which comprises administering to a subject in need thereof an effective amount of such agent or composition thereof. The compositions of the present invention are useful in the management of hair disease(s)/disorder(s) including prophylaxis, amelioration or treatment of such hair disease(s)/disorder(s).

In a further embodiment, the hair loss preventing and/or hair growth promoting agent or compositions thereof is useful in one or more of several hair disorders including but not limited to a group comprising alopecia greata, androgenetic alopecia, anagen effluvium (cancer treatment hair loss), self induced hair loss, telogen effluvium, scarring alopecia, syphilitic alopecia, scleroderma and tinea capitis. In an embodiment, the hair loss preventing and/or hair growth promoting active agent preferably acts by blocking or inhibiting the Testosterone 5α-reductase responsible for converting testosterone to 5α-DHT. The 5α-DHT causes the hair shafts to narrow producing progressively finer hairs with each new growth cycle until eventually the hairs become transparent and stop emerging. Thus this male hormone, 5α-DHT contributes to androgenetic alopecia primarily in those who are genetically predisposed.

In a further embodiment, the hair loss preventing and/or hair growth promoting active agent of the present invention is additionally combined with one or more allopathic drugs that are available to treat alopecia, occurring due to different pathological conditions, such as 5α-DHT inhibitors (Anti-androgens), SOD (Super oxide dismutases) minetics, Vasodilators, Activation of PGHS-1 (Prostaglandin-H synthase-1) and Potassium channel openers (PCOs). Preferably such combination leads to an additive, potentiating or a synergistic effect and might lead to a reduction in the dose of the allopathic drug used, thus minimizing the dose dependent adverse effects associated with such drug. In a further embodiment, the hair loss preventing and/or hair growth promoting active agent of the present invention is additionally combined with one or more allopathic drugs selected from but not limited to a group comprising corticosteroids, dithranol, retin A (tretinoin), minoxidil, zinc, irritants, finasteride, skinoren/azelaic acid, cyproterone acetate with ethinyloestradiol, cimetidine, cyproterone acetate, spironolactone, ketoconazole, antidepressant, triamcinolone acetonide, antimalarial drugs such as hydroxychloroquine, penicillin, and the like, or mixtures thereof. Any other suitable drug known to the art that is useful for treatment of hair disorders or other associated disorders such as depression or anxiety due to hair loss, etc. can be combined with the hair loss preventing and/or hair growth promoting active agent of the present invention.

In a yet another embodiment, the novel compositions of the present invention can be formulated as a cosmetic, herbal, ayurvedic or pharmaceutical dosage form known to the art, preferably in the form of an oral preparation such as tablets or capsules or a topical preparation such as liquid, cream, gel, lotion or spray that are useful for hair loss prevention and/or hair growth promotion preferably for the treatment of testosterone induced androgenic alopecia. The compositions may also be in the form of a shampoo or conditioner or hair oil that could be applied topically at the desired site. In another embodiment, the preferred dose of the hair loss preventing and/or hair growth promoting active agent of the present invention is approximately about 0.01% to about 15.0% w/w, preferably about 0.1% to about 5.0% w/w of the composition.

### Pharmacological Studies

The efficacy of an extract of Vernonia anthelmintica (prepared according to Example-1 as stated hereinafter) against testosterone-induced alopecia in hamsters was studied. Male hamsters (n=6/group) weighing 90-120 g were procured from central animal house of Panacea Biotec Ltd. for study. Drugs used to induce alopecia was Testosterone i.m. depot injection (Testoviron); B.No. K1007, German Remedies Limited; each ml of which contains Testosterone Enanthate USP . . . 250 mg and Arachis oil IP . . . qs. The route of administration of the cream compositions was topical and the duration of study was 22 days. Hamsters were divided into two different groups; one Vernonia anthelmintica extract treated groups and another testosterone control group. On day 0 of study, the fur over and around the flank organs would be shaved with electric clippers. The different treatments are summarized in Table-1.

TABLE 1
Summary of treatment schedule

| Group No. | Treatment |
|---|---|
| I | Testosterone (25 mg in divided volume of |
| | 33.3 μl on day 0, 7 and 14) + |
| | 95% v/v alcohol (100 μl/site) |
| II | Vernonia anthelmintica extract (100 μl/site) |

Testosterone was administered intramuscularly in divided doses, whereas Vernonia anthelmintica extract was applied topically twice daily for 21 days. On day 7, the fur around the flank organs was re-clipped. The amount of hair growth on the area surrounding the flank organs was visually graded on day 22 on a 0-3 scale (0=bald skin, 1=slight hair growth, 2=moderate hair growth and 3=full hair growth). Photographs of flank organ were taken on day 0 and 22. On day 22, testosterone-treated hamsters showed a significant hair loss as compared to normal group (Hair growth score; testosterone-treated=0.58±0.15 as compared to control=2.0). Treatment with Vernonia anthelmintica extract (topical, 100 μl/site) significantly reversed testosterone induced-hair loss in hamsters (FIG. 1). The representative photographs from different treatment groups are shown in FIG. 2. The ‘Control’ group showed normal hair growth on day 22, which was prevented by testosterone treatment. Further, treatment with extract of Vernonia anthelmintica for 22 days reversed testosterone-induced hair loss.

Another study was conducted in hamsters to demonstrate the comparative efficacy of an extract of Vernonia anthelmintica (prepared according to Example-2 as stated hereinafter) against finasteride in androgen induced alopecia. Hamsters were divided into five different groups; one normal control group, three drug treated groups and one testosterone control group. On day 0 of the study, the fur over and around the flank organs was shaved with electric clippers. 95% alcohol (100 μl/site) was applied topically, twice daily to Testosterone Control and Normal control groups for 21 days. Saw Palmetto cream was applied topically (100 mg/site) twice daily for 21 days. 100 μl/site of 2% solution of Vernonia anthelmintica extract was applied topically, twice daily for 21 days. Finasteride (0.6 mg/kg) was administered orally twice daily for 21 days. Different groups except normal control group was administered testosterone (25 mg, i.m. in divided doses of 33.3 μl on day 0, 7, and 14) 1-hour post drug administration. At day 7, the fur around the flank organs was re-clipped. The amount of hair growth on the area surrounding the flank organs was visually graded at weekly intervals on a 0-3 scale (0=bald skin, 1=slight hair growth, 2=moderate hair growth and 3=full hair growth). The result is presented in table-2 and shown graphically in FIG. 3. Representative photographs of hair growth in hamsters for different treatments are shown in FIG. 4. The control group showed normal hair growth which was prevented by testosterone treatment. Further, finasteride or Vernonia anthelmintica extract composition but not Saw palmetto significantly reversed testosterone induced-hair loss as compared to control group on day 22. The study indicated that the extract of Vernonia anthelmintica is significantly more effective than finasteride in the treatment of testosterone induced androgenic alopecia.

TABLE 2
Comparative hair growth profile of an extract
of Vernonia anthelmintica against finasteride

| S. No. | Treatment | Mean* ± SEM |
|---|---|---|
| 1. | Control | 2.75 ± 0.171 |
| 2. | Testosterone (33.3. μl) | 1.33 ± 0.211 |
| 3. | Saw Palmetto (100 mg) | 1.58 ± 0.201 |
| 4. | Vernonia anthelmintica extract | 2.83 ± 0.167 |
| | 2% w/v (100 μl) | |
| 5. | Finasteride (0.6 mg/kg) | 2.20 ± 0.339 |

*Hair growth on a 0-3 scale (0 = bald skin, 1 = slight hair growth, 2 = moderate hair growth and 3 = full hair growth).

A further study was conducted in order to determine the strength of topical composition comprising Vernonia anthelmintica extract particularly in the form of Vernonia creams which exhibit hair growth promoting effect in testosterone-induced alopecia in hamsters. Male hamsters (n=6/group) weighing 80-90 g procured from Central animal house of Panacea Biotec Ltd. were used. Drugs used to induce alopecia was Testosterone i.m. depot injection (Testoviron); B.No. K1007, German Remedies Limited; each ml of which contains Testosterone Enanthate USP . . . 250 mg and Arachis oil IP . . . qs. Four batches of Vernonia creams of different strengths were used for conducting the study namely B.No. 0629/010A (2% w/w), B.No. 0629/010C (0.2% w/w), B.No. 0629/014A (2% w/w) and B.No. 0629/014C (0.2% w/w) prepared according to Example-3 and Example 4 as stated hereinafter. The route of administration of the cream compositions was topical and the duration of study was 22 days. The fur over and around the flank organs of hamsters was shaved with electric clippers. Hamsters were divided into four different groups and allocated different treatments. The summary of different treatments is represented in Table-3.

TABLE 3
Summary of treatment schedule

| Group No. | Treatment |
|---|---|
| I | Control |
| II | Testosterone (25 mg in divided volume |
| | of 33.3 μl on day 0, 7 and 14) |
| III | B. No. 0629/010A, 2% w/w; 100 mg, bid |
| IV | B. No. 0629/010C, 0.2% w/w; 100 mg, bid |
| V | B. No. 0629/014A, 2% w/w; 100 mg, bid |
| VI | B. No. 0629/014C, 0.2% w/w; 100 mg, bid |

Testosterone was administered intramuscularly in divided doses, whereas Vernonia cream(s) (100 mg) was applied topically twice daily. All the treatments were given for 21 days. On day 7, the fur around the flank organs was re-clipped. The amount of hair growth on the area surrounding the flank organs was visually graded on day 22 on a 0-3 scale (0=bald skin, 1=slight hair growth, 2=moderate hair growth and 3=full hair growth). Photographs of flank organs for each treatment were taken before any treatment and at day 22 to study the hair growth changes, which are shown in FIG. 5. The hair growth presented as mean was analyzed by one-way ANOVA followed by Student-Newman-Keuls multiple-range test. P<0.05 was accepted as the level of significant difference. On day 22, testosterone-treated hamsters showed a significant hair loss as compared to normal group (FIGS. 5(a) and 5(b)) (Hair growth score; testosterone-treated 0.13 vs. normal control=3). The topical application of Vernonia cream 2% w/w (B.No. 06291010A) for 21 days significantly prevented testosterone induced-hair loss in hamsters (hair growth score 2.08) when compared to testosterone group (hair growth score 0.13) (FIGS. 5(c) and 5(b), respectively). Comparatively lesser hair growth was observed in hamsters treated with other Vernonia creams (B.No. 0629/010C, B.No. 0629/014A and B.No. 0629/014C). Hair growth score for B.NQ. 0629/010C was 0.67, B.No. 0629/014A was 0.33, and B.No. 0629/014C was 0.17 as compared to 0.13 for testosterone group (FIGS. 5(d), 5(e), 5(f) and 5(b) respectively). The hamsters treated with B.No. 0629/014A and B.No. 0629/014C and their respective placebo developed scales over the area of application (FIGS. 5(e), 5(f) and 5(g) respectively). No scale formation over the area of application was observed in hamsters treated with Vernonia creams B.No. 0629/010A and B.No. 0629/010C or their placebo (FIGS. 5(c), 5(d) and 5(h)). Control group showed normal hair growth which was prevented by testosterone treatment. Further, Vernonia cream (2% w/w, B.No.0629/10A) significantly prevented testosterone induced-hair loss as compared to testosterone group. However, other cream formulations of Vernonia cream prevented the testosterone-induced hair loss to a lesser extent. The results of the present study suggested that the Vernonia cream of B.No. 0629/010A i.e. 2% strength composition was comparatively more effective in promoting hair growth in testosterone-challenged hamsters as compared to the other compositions studied.

A further study was performed to study the dose-response of Vernonia extract gel and to compare its hair growth promoting effect against Vernonia freeze dried juice gel. The Vernonia extract gel was prepared by making an organic solvent extract of Vernonia and formulating it into a gel composition. The Vernonia freeze dried juice gel was prepared by expressing the juice from fresh leaves and flowering tops of Vernonia followed by freeze drying and formulating it into a gel composition. Male hamsters (n=6-8/group) weighing 80-90 g procured from the Central animal house of Panacea Biotec Ltd. were used. Drugs used to induce alopecia was Testosterone i.m. depot injection (Testoviron); B.No. K1007, German Remedies Limited; each ml of which contains Testosterone Enanthate USP . . . 250 mg and Arachis oil IP . . . qs. The following were used for conducting the study namely Vernonia extract gels B.No. 0629/030A (0.5% w/w), B.No. 0629/030B (1% w/w) and B.No. 0629/030C (2% w/w); and Vernonia freeze dried juice gels B.No. 0629/026A (1.25% w/w) and B.No. 0629/026B (2.5% w/w) (prepared according to Example-5 and Example-6 as stated hereinafter). The route of administration of the compositions was topical and the duration of study was 22 days. The fur over and around the flank organs of hamsters was shaved with electric clippers. Hamsters were divided into four different groups and allocated different treatments. The summary of different treatments is represented in Table-4.

TABLE 4
Summary of treatment schedule

| Group No. | Treatment |
|---|---|
| I | Control |
| II | Testosterone (25 mg in divided volume |
| | of 33.3 μl on day 0, 7 and 14) |
| III | B. No. 0629/030A, 0.5% w/w; 100 mg, bid |
| IV | B. No. 0629/030B, 1% w/w; 100 mg, bid |
| V | B. No. 0629/030C, 2% w/w; 100 mg, bid |
| VI | B. No. 0629/026A 1.25% w/w; 100 mg, bid |
| VII | B. No. 0629/026B 2.5% w/w; 100 mg, bid |

Testosterone was administered intramuscularly in divided doses, whereas Vernonia cream (s) (100 mg) was applied topically twice daily. All the treatments were given for 21 days. On day 7, the fur around the flank organs was re-clipped. The amount of hair growth on the area surrounding the flank organs was visually graded on day 22 on a 0-3 scale (0=bald skin, 1=slight hair growth, 2=moderate hair growth and 3=full hair growth). Photographs of flank organs were taken for each treatment before any treatment and at day 22 to study the hair growth changes. The hair growth presented as mean was analyzed by one-way ANOVA followed by Student-Newman-Keuls multiple-range test. P<0.05 was accepted as the level of significant difference. The scores and representative photographs of hair growth in hamsters are represented in FIG. 6 and FIG. 7 respectively. On day 22, testosterone-treated hamsters showed complete hair loss as compared to normal group (Hair growth score; testosterone-treated 0 vs. normal control=1.31±0.18) (FIGS. 7(b) and (d)). Animals in the control group showed normal hair growth (FIG. 6(b)). The topical application (100 mg, bid) of Vernonia extract gels (0.5, 1, and 2% w/w) for 21 days significantly prevented testosterone induced-hair loss in hamsters when compared to testosterone group (FIG. 6, FIGS. 7(e), (f) and (g)). The effect was found to be dose-dependent. Further, Vernonia extract gels (1 and 2% w/w)-induced hair growth (hair growth score) was found to be more intense than the normal hair growth in control group. (FIG. 6). Similarly, the Vernonia freeze dried juice gels (1.25 and 2.5% w/w) also significantly promoted hair growth in testosterone-challenged hamsters (FIG. 6, FIGS. 7(h) and (i)). The hair growth was found to be dose-dependent, yet significantly lower than that of all the extract gels and normal control as well (FIG. 6). The results of the study demonstrated a dose-dependent hair growth promoting effect in hamsters treated with Vernonia extract gel (0.5-2% w/w), which at dose level 1% and 2% w/w was more intense than normal hair growth. Likewise, the Vernonia freeze dried juice extracts (1.25% and 2.5% w/w) also demonstrated a dose dependent effect on hair growth in hamsters but less than that of extract gels or normal hair growth.

### BRIEF DESCRIPTION OF FIGURES

FIG. 1: Effect of Vernonia anthelmintica extract (topical, 100 μl/site) on testosterone-induced hair loss in hamsters. Data is represented as mean±S.E.M. *P<0.05 as compared to testosterone treated group.

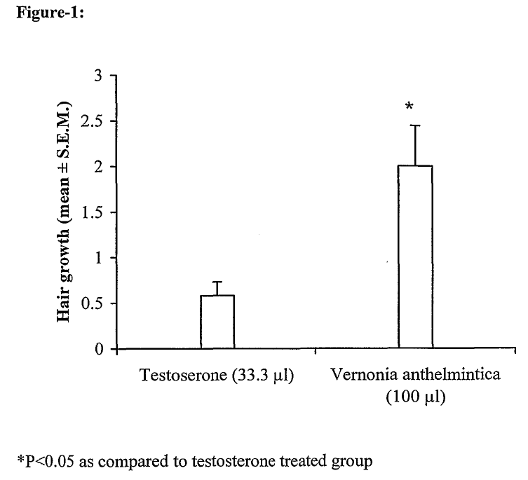

FIG. 2: Representative photographs of hair growth in hamsters: (a) control group (day 0), (b) control group day 22, (c) testosterone-treated (day 22) and (d) Vernonia anthelmintica extract-treated.

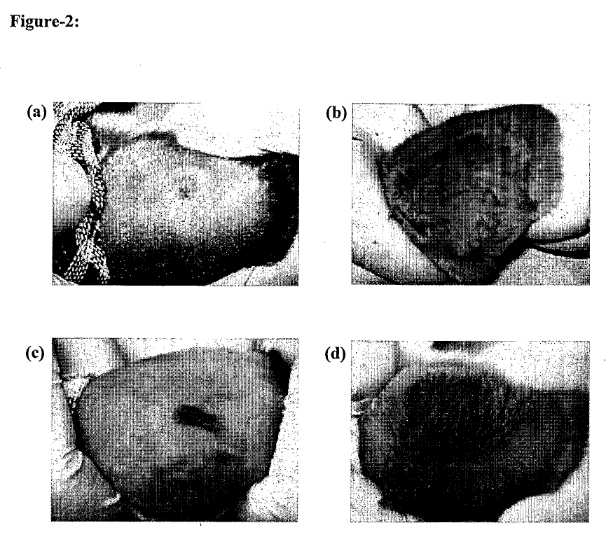

FIG. 3: Comparative hair growth profile of extract of Vernonia anthelmintica against finasteride.

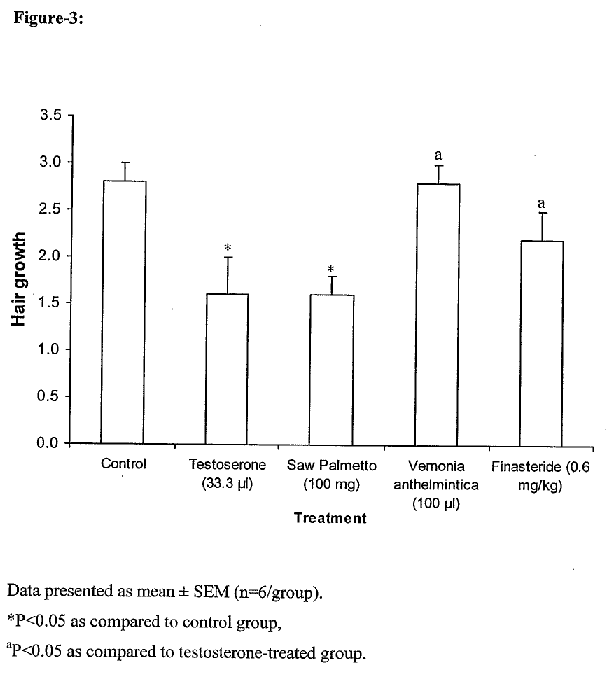

FIG. 4: Representative photographs of hair growth in hamsters: (a) control group day 0, (b) control group day 22, (c) testosterone-treated day 22, (d) Saw palmetto day 22, (e) Arnebia euchroma day 22, (f) Vernonia anthelmentica day 22 and (g) finasteride day 22.

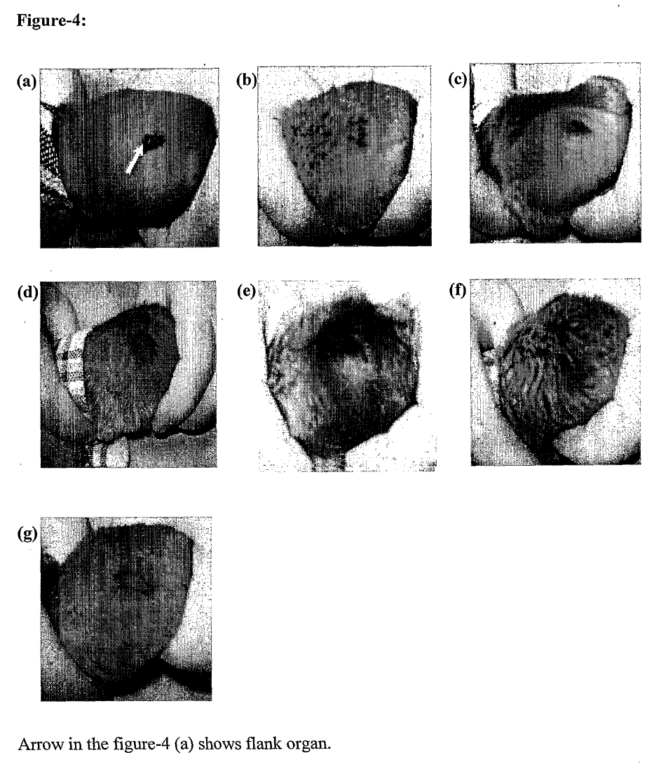

FIG. 5: Representative photographs of hair growth on day 22 in hamsters: (a) control group, (b) testosterone-treated, (c) treated with Vemonia cream 2% w/w, B.No. 0629/10A, (d) treated with Vernonia cream 0.2% w/w, B.No. 0629/10C, (e) treated with Vernonia cream 0.2% w/w, B.No. 0629/14A and (f) treated with Vernonia cream 0.2% w/w, B.No. 0629/14C.

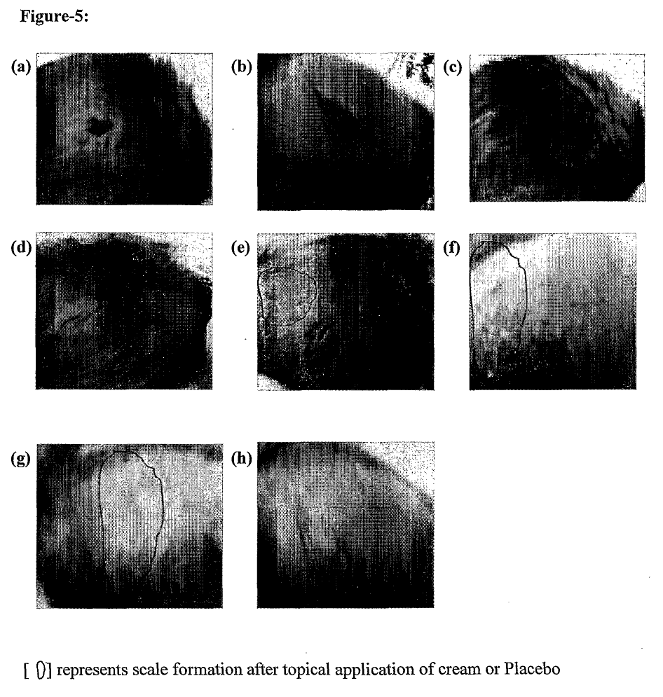

FIG. 6: Relative profile of hair growth score in hamster treated with Vernonia extract gel (0.5-2% w/w) or Vernonia freeze dried juice gel (1.25 and 2.5% w/w) (n=6-8).

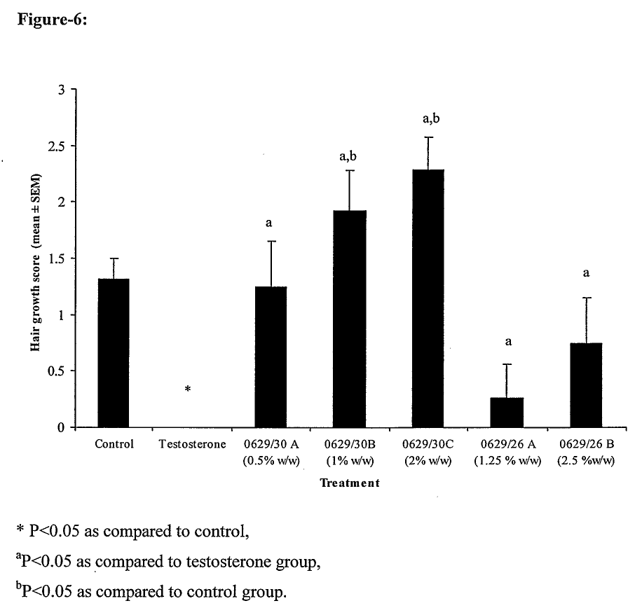

FIG. 7: Representative photographs of hair growth pattern in hamsters: (a) control group (day 0), (b) control group (day 22), (c) testosterone-treated (day 0), (d) testosterone-treated (day 22), (e) 2% w/w, B.No. 0629/30C, (f) 1% w/w, B.No. 0629/30B, (g) 0.5% w/w, B.No. 0629/30A, (h) 2.5% w/w, B.No. 0629/26B and (i) 1.25% w/w, B.No. 0629/26A.

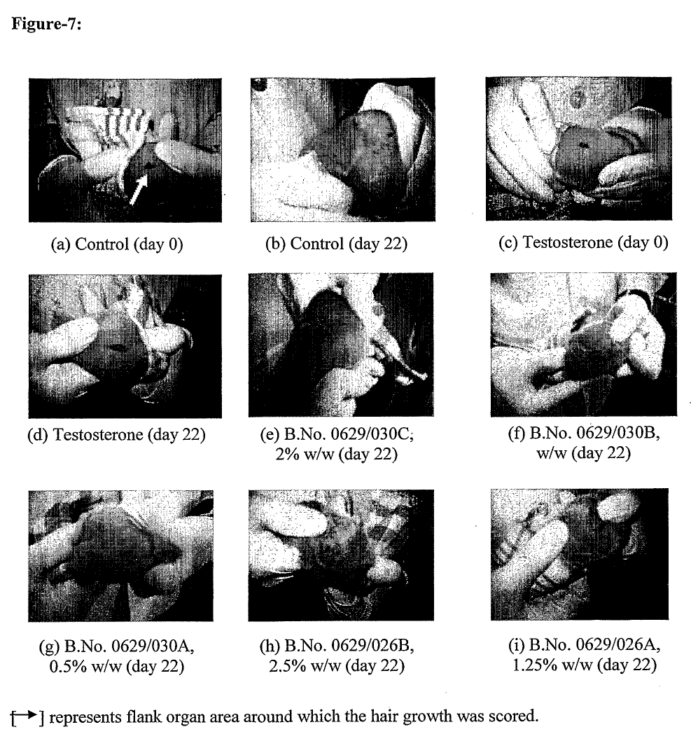

In an embodiment, the carrier useful in the present invention is selected from but not limited to a group comprising monosaccharides, disaccharides, polysaccharides, sugar alcohols, polylactic acid, cyclodextrin, lactose, glucose, raffinose, melezitose, xylitol, arabinose, dextran, lactitol, maltitol, trehalose, sucrose, mannitol and starch, and the like or mixtures thereof.

In a further embodiment, the pharmaceutical composition of the present invention further comprises one or more pharmaceutically acceptable excipient(s) selected from but limited to the group comprising diluents, disintegrants, binders, anti-adherants, glidants, anti-oxidants, buffering agents, colorants, flavoring agents, coating agents, solvents, viscosifying agents, waxes, wetting agents, emulsifying agents, solubilizers, stabilizers, buffering agents, vehicles, preservatives, surfactants, deodorants, colorants, and the like.

The pharmaceutical compositions of the present invention can be prepared by dissolving or dispersing the extract of Vernonia sp. in appropriate base(s)/carrier(s) known to the art. The pharmaceutical composition into different dosage forms can be formulated using conventional excipients and techniques known to art. Pharmaceutical dosage forms of the present invention can be creams, ointment, gels, foams, solutions, suspensions, medicated pad, powder, aerosols, sprays, film, and flakes. The compositions can be formulated as immediate release dosage forms or modified release dosage forms (sustained release, extended release, delayed release, prolonged release, timed release, pulsatile release and the like) or combination of such forms. The pharmaceutical compositions of the present invention comprise the extract of the plant Vernonia sp. from about 0.01% to about 99% by weight along with one or more carrier(s) from about 1% to about 99.99% by weight of the composition optionally along with one or more excipient(s).

The cream composition comprising the extract of the plant Vernonia sp. is prepared by emulsifying the aqueous phase, comprising about 0.1-10% w/w preferably about 0.2-5% w/w of the extract, along with a suitable oleaginous phase. Other alternatives can be prepared by formulating the extract in about 0.1-10% w/was Hydrophilic ointment USP with absorption bases; or water soluble bases such as Polyethylene glycol ointment USNF; or as water absorbing bases such as Hydrophilic petrolatum USP, Lanolin USP; or in hydrocarbon bases such as White petrolatum USP. Other hydrophobic or hydrophilic base that are useful includes cocoa butter, glycerinated gelatin, hydrogenated vegetable oils, mixtures of polyethylene glycols of various molecular weights, polyoxyethylene sorbitan fatty acid esters and polyethylene stearates, polyvinyl alcohol, polyvinyl pyrrolidone, polyacrylamide, chemically modified starch or a combination of these materials. In another embodiment, the hydrocarbon base comprises paraffins, waxes, petroleum jelly, lanolin, and the like or mixtures thereof. The foam and/or spray base comprises one or more of aqueous and nonaqueous solvents, propellants, surfactants, suspending agents and stabilizing agents. The medicated pads comprise one or more of the following: Water, glycerin, propylene glycol, alcohol and Hamamelis water and the like.

In another embodiment, the compositions of the present invention additionally comprises hygroscopic moisturizers (humectants) such as polyhydric alcohols, sodium 2-pyrrolidone-5-carboxylate (NaPCA), amino acids and derivatives, guanidine; glycolic acid and glycolate salts (e.g. ammonium and quaternary alkyl ammonium); lactic acid and lactate salts (e.g. ammonium and quaternary alkyl ammonium); other alpha hydroxy acids such as malic acid, aloe vera in any of its variety of forms (e.g. aloe vera gel); hyaluronic acid, precursors and derivatives thereof (e.g. glucosamine and salt derivatives such as sodium hyaluronate); lactamide monoethanolamine; acetamide monoethanolamine; urea; and mixtures thereof. Preferred occlusive moisturizers for use herein are petrolatum, isohexadecane, isononyl isononanoate, methyl isostearate, isopropyl isostearate, and mixtures thereof. According to an embodiment, the gelling agents or gel-forming agents useful in the present invention, which possess adequate mechanical, physiological and release properties, are preferably polysaccharides, like alginates, pectins, carrageenans or xanthan, starch and starch derivatives, gums like tragacanth or xanthan gum, collagen, gelatin, galactomannan and galactomannan derivatives, chitosan and chitosan derivatives, glycoproteins, proteoglycans, glucosaminoglycans, polyvinyl alcohol, polyvinylpyrrolidone, vinylpyrrolidone/vinyl acetate copolymers, high molecular weight polyethylene glycols and/or high molecular weight polypropylene glycols, polyoxyethylene/polyoxypropylene copolymers, polyvinyl alcohol, polyacrylates and/or polymethacrylates, polylactides, polyglycolides and polyamino acids, and cellulose derivatives. Especially preferred gel-forming agents are selected from cellulose derivatives, especially cellulose ether compounds, like methylcellulose, hydroxypropyl cellulose, hydroxyethyl cellulose, hydroxypropyl methylcellulose, sodium carboxymethylcellulose, ethyl cellulose, cellulose acetate phthalate, hydroxypropyl methylcellulose phthalate, cellulose acetate succinate and ethyl cellulose succinate. The carrier may contain one or more additional excipient(s) like sugars, sugar alcohols, surfactants, amino acids, antioxidants, polyethylene glycols, and the like.

In an embodiment, the carrier may be a vegetable or a mineral oil or a combination of both. In an embodiment, the vegetable oil useful in the present invention is selected from but not limited to a group comprising sunflower oil, soyabean oil, linseed oil, cottonseed oil, olive oil, palm oil, coconut oil, sesame oil, safflower oil, and the like or mixtures thereof. Additional substances such as yohimbine (selective competitive alpha2-adrenergic receptor antagonist for local vasodilation), clove oil (mild stimulant and local anesthetic), arginine (capillary blood circulation enhancer), and the like or mixtures thereof can be added to the preparation.

Other acceptable carriers useful in formulating the compositions of the present invention are preferably lubricants such as carboxymethyl cellulose, sodium alginate, EDTA, natural vegetable oils, propylene glycol, glycerin, low melting temperature triglyceride, mineral oil, aqueous solutions of high molecular weight polyethylene oxides, and the like or mixtures thereof. Polymers such as water soluble cellulose derivative (e.g. methyl cellulose, hydroxyethyl cellulose, hydroxypropyl cellulose, sodium carboxymethyl cellulose) or other water soluble polymers such as sodium alginate, polyvinyl pyrrolidone, polyvinyl alcohol or polymer of ethylene oxide, and the like can be used in the present invention. Other optional substances that can be used in the present invention include anti-infectives such as parabens, chlorhexidine, benzyl alcohol, and the like.

Further, the acceptable conventional excipients useful in the composition of the present invention are selected from but not limited to a group of excipients generally known to persons skilled in the art such as fillers, binders, lubricants, colorants; stabilizers; preservatives; chelating agents; vehicles; bulking agents; hydrophilic polymers; solubility enhancing agents such as glycerine, various grades of polyethylene oxides, transcutol and glycofurol; tonicity adjusting agents; local anesthetics; pH adjusting agents; antioxidants; osmotic agents; chelating agents; viscosifying agents; wetting agents; emulsifying agents; acids; sugar alcohol; reducing sugars; non-reducing sugars and the like used either alone or in combination thereof e.g. diluents such as lactose, mannitol, sorbitol, starch, microcrystalline cellulose, xylitol, fructose, sucrose, dextrose, dicalcium phosphate, calcium sulphate; bulking agent and organic acid(s). The lubricants used in the present invention include but not limited to talc, magnesium stearate, calcium stearate, stearic acid, hydrogenated vegetable oil and the like used either alone or in combination thereof. The vehicles suitable for use in the present invention can be selected from but not limited to a group comprising dimethylacetamide, dimethylformamide and dimethylsulphoxide of N-methyl pyrrolidone, benzyl benzoate, benzyl alcohol, ethyl oleate, polyoxyethylene glycolated castor oils (Cremophor® EL), polyethylene glycol MW 200 to 6000, propylene glycol, hexylene glycols, butylene glycols and glycol derivatives such as polyethylene glycol 660 hydroxy stearate (commercially available as Solutrol® HS15). In another embodiment of the present invention, the compositions may additionally comprise an antimicrobial preservative such as Benzyl alcohol preferably at a concentration of about 2.0% v/v of the composition. In an embodiment of the present invention, the composition may additionally comprise a conventionally known antioxidant such as ascorbyl palmirate, butyl hydroxy anisole, butyl hydroxy toluene, propyl gallate and α-tocopherol. In another embodiment, additionally surfactants including ionic and non-ionic surfactants, sorbitan esters such as sorbitan trioleate, sorbitan monooleate, sorbitan monolaurate, Polyoxyethylene sorbitan esters such as polyoxyethylene sorbitan monolaurate, polyoxyethylene sorbitan monooleate, poloxamer, fluorinated and non-fluorinated surfactants, carboxylic acids, polyethoxylates, natural lecithin, oleyl polyoxyethylene ether, stearyl polyoxyethylene ether, lauryl polyoxyethylene ether, block copolymers of oxyethylene and oxypropylene, synthetic lecithin, diethylene glycol dioleate, tetrahydrofurfuryl oleate, ethyl oleate, glyceryl monooleate, polyethylene glycol 400 and glyceryl monolaurate and the like or mixtures thereof are used in the composition. The topical compositions of the present invention may comprise a wide variety of further optional components; provided that such optional components are physically and chemically compatible with the essential components described herein, and do not unduly impair stability, efficacy or other use benefits associated with the compositions of the present invention.

The compositions of the present invention and method for treating hair disorders or other associated disorders using an extract of Vernonia sp. provides long-term effectiveness, high rate of hair regeneration and/or low rates of hair loss. The treatment includes administration of an effective amount of composition comprising of an extract of Vernonia sp. and a carrier(s), preferably as a local application at the desired site of action.

In an embodiment of the present invention, aerosol for topical spray is prepared comprising the Vernonia extract as active agent(s) preferably in the micronized form along with a suitable vehicle or a propellant system preferably dispensed into aluminium containers sealed with metering valves by means of the pressure-filling technique. Nebulizable dispersions or solutions for atomization are prepared by dispersing the active agent(s) homogeneously in a hydro-alcoholic solvent system such as ethanol-purified water mixture. Suspensions for local application are prepared by wetting the active agent(s) with a wetting agent such as surfactant followed by addition of optionally other excipient(s), filling the bulk into sterile containers, for example unit dose containers such as containers which are suitably molded from thermoplastics.

The examples given below serve to illustrate embodiments of the present invention. However they do not intend to limit the scope of present invention.

### EXAMPLES

### Example-1

The preparation of extract for hair loss prevention and/or hair growth promotion from the plant Vernonia sp. comprises of the following steps:

1. 2 kg of dried and powdered plant is added to 5 L of Chloroform in a flask.

2. The mixture is boiled under a reflux condenser for 1 hour.

3. The mixture is cooled and filtered. The filtrate is set aside.

4. Steps 1 to 3 are repeated with the residue three times more.

5. The pooled filtrates are distilled to remove hexane.

### Example-2

The preparation of extract for hair loss prevention and/or hair growth promotion from the plant Vernonia sp. comprises of the following steps:

1. 1 kg of dried and powdered leaves is added to 6 L of Hexane in a flask.

2. The mixture is boiled under a reflux condenser for 1 hour.

3. The mixture is cooled and filtered. The filtrate is set aside.

4. Steps 1 to 3 are repeated with the residue three times more.

5. The pooled filtrates are distilled to remove hexane.

6. The hexane extract is stirred with 500 ml 95% ethanol for 30 minutes.

7. The ethanolic mixture is filtered. The filtrate is set aside.

8. Steps 6 and 7 are repeated with the residue.

9. The pooled ethanolic extracts are distilled to remove ethanol

### Example-3

The preparation of extract for hair loss prevention and/or hair growth promotion from the plant Vernonia sp. comprises of the following steps:

1. 2 kg of dried and powdered plant is added to 5 L of water.

2. The mixture is boiled under a reflux condenser for 1 hour.

3. The mixture is cooled and filtered. The filtrate is set aside.

4. Steps 1 to 3 are repeated with the residue once more.

5. Pooled filtrates are concentrated and spray dried.

### Example-4

The preparation of extract for hair loss prevention and/or hair growth promotion from the plant Vernonia sp. comprises of the following steps:

1. 2 kg of dried and powdered plant is added to 10 L of ethanol.

2. The mixture is boiled under a reflux condenser for 1 hour.

3. The mixture is cooled and filtered. The filtrate is set aside.

4. Steps 1 to 3 are repeated with the residue for three times more.

5. Pooled filtrates are concentrated and dried in an agitated vacuum drier.

### Example-5

The preparation of extract for hair loss prevention and/or hair growth promotion from the plant Vernonia sp. comprises of the following steps:

1. 5 kg of fresh plant is crushed in a mixer/grinder with addition of 1 L of water.

2. The juice of the mixture is expressed out and filtered.

3. The filtrate is freeze dried.

In the examples stated below describing composition comprising the novel hair loss preventing and/or hair growth promoting extract of the present invention, the quantity stated may be varied based on the desired preventive or ameliorative or therapeutic effect and the type of composition.

### Example-6

### Vernonia Cream

| | | % w/w | |
|---|---|---|---|
| S. No. | Ingredient | 0629/010A | 0629/010C |
| 1. | Vernonia anthelmentica extract | 2.0 | 0.2 |
| 2. | Carbomer 940 | 0.6 | 0.6 |
| 3. | Purified Water | 62.1 | 63.9 |
| 4. | Transcutol ® (Diethylene | 20.00 | 20.00 |
| | Glycol Monoethyl Ether) | | |
| 5. | Ethanol | 10.00 | 10.00 |
| 6. | Menthol | 0.30 | 0.30 |
| 7. | Propylene Glycol | 5.00 | 5.00 |
| 8. | Triethanolamine | q.s. | q.s. |

Procedure:
i) Disperse Carbomer 940 in Purified Water and stir till it dissolves completely.
ii) Dissolve Menthol in mixture of Ethanol and Transcutol ®. Add Propylene Glycol and Vernonia anthelmentica extract into it and mix.
iii) Add bulk of step (ii) into bulk of step (i) and mix.
iv) Add Triethanolamine drop wise into the bulk of step (iii) until pH is in the range of 5.5-7.0 and a semisolid gel is obtained.

### Example-7

### Vernonia Cream

| | | % w/w | |
|---|---|---|---|
| S. No. | Ingredient | 0629/014A | 0629/014C |
| 1. | Vernonia anthelmentica extract | 2.00 | 0.20 |
| 2. | Carbomer 940 | 0.60 | 0.60 |
| 3. | Purified Water | 56.35 | 58.15 |
| 4. | Transcutol ® (Diethylene | 20.00 | 20.00 |
| | Glycol Monoethyl Ether) | | |
| 5. | Ethanol | 10.00 | 10.00 |
| 6. | Menthol | 0.30 | 0.30 |
| 7. | Perfume | q.s. | q.s. |
| 8. | Triethanolamine | q.s. | q.s. |
| 9. | Oleic Acid | 5.50 | 5.50 |
| 10. | Polyoxyethylene (2) Stearyl Ether | 1.00 | 1.00 |
| | (Brij ® 72) | | |
| 11. | Cetyl Alohol | 0.75 | 0.75 |
| 12. | Vitamin E Acetate | 0.50 | 0.50 |
| 13. | Isopropyl Myristrate | 3.00 | 3.00 |

Procedure:
i) Disperse Carbomer 940 in Purified Water and stir till it dissolves completely.
ii) Dissolve Menthol in mixture of Ethanol and Transcutol ®.
iii) Add bulk of step (ii) into bulk of step (i) and mix.
iv) In separate beaker, add Oleic Acid, Brij ® 72, Cetyl Alcohol, Vitamin E Acetate and Vernonia anthelmentica extract and mix.
v) Heat the bulk of step (iii) and (iv) separately at 60-70° C.
vi) Add oil phase of step (iv) into aqueous phase of step (iii) with constant stirring.
vii) Add Triethanolamine drop wise into the bulk of step (vi) until pH is in the range of 6.0-7.0 and a semisolid cream is obtained.
viii) Add perfume to the semisolid cream and mix to obtain the desired product.

### Example-8

### Vernonia Freeze Dried Juice Gel

| | | % w/w | |
|---|---|---|---|
| S. No. | Ingredient | 0629/026A | 0629/026B |
| 1. | Vernonia anthelmentica extract | 1.25 | 2.50 |
| | freeze dried powder | | |
| 2. | Carbomer 940 | 1.50 | 1.50 |
| 3. | Purified Water | 61.95 | 60.70 |
| 4. | Transcutol ® (Diethylene | 20.00 | 20.00 |
| | Glycol Monoethyl Ether) | | |
| 5. | Ethanol | 10.00 | 10.00 |
| 6. | Menthol | 0.30 | 0.30 |
| 7. | Propylene Glycol | 5.00 | 5.00 |
| 8. | Perfume | q.s. | q.s. |
| 9. | Triethanolamine | q.s. | q.s. |

Procedure:
i) Disperse Carbomer 940 in Purified Water and stir till it dissolve completely.
ii) Dissolve Vernonia anthelmentica extract powder in part of Purified Water and add it to the bulk of step (i).
iii) Dissolve Menthol in mixture of Ethanol and Transcutol ®. Add Propylene Glycol into it and mix.
iv) Add bulk of step (iii) into bulk of step (ii) and mix.
v) Add Triethanolamine drop wise into the bulk of step (iv) until pH is in the range of 5.5-7.0 and a semisolid gel is obtained.
vi) Add perfume to the semisolid gel and mix.

### Example-9

### Vernonia Extract Gel

| | | % w/w | | |
|---|---|---|---|---|
| S. No. | Ingredient | 0629/030A | 0629/030B | 0629/030C |
| 1. | Vernonia | 0.5 | 1.0 | 2.0 |
| | anthelmentica | | | |
| | extract | | | |
| 2. | Carbomer 940 | 0.75 | 0.75 | 0.75 |
| 3. | Purified Water | 63.45 | 62.95 | 61.95 |
| 4. | Transcutol ® | 20.00 | 20.00 | 20.00 |
| | (Diethylene Glycol | | | |
| | Monoethyl Ether) | | | |
| 5. | Ethanol | 10.00 | 10.00 | 10.00 |
| 6. | Menthol | 0.30 | 0.30 | 0.30 |
| 7. | Propylene Glycol | 5.00 | 5.00 | 5.00 |
| 8. | Perfume | q.s. | q.s. | q.s. |
| 9. | Triethanolamine | q.s. | q.s. | q.s. |

Procedure:
i) Disperse Carbomer 940 in Purified Water and stir till it dissolves completely.
ii) Dissolve Menthol in mixture of Ethanol and Transcutol ®. Add Propylene Glycol and Vernonia anthelmentica extract into it and mix.
iii) Add bulk of step (ii) into bulk of step (i) and mix.
iv) Add Triethanolamine drop wise into the bulk of step (iii) until pH is in the range of 5.5-7.0 and a semisolid gel is obtained.
v) Add perfume to the semisolid gel and mix.

### Example-10

### Vernonia Cream

| S. No. | Ingredient | % w/w |
|---|---|---|
| 1. | Vernonia cinerea extract | 1.00 |
| 2. | Light liquid paraffin | 8.00 |
| 3. | White Bees Wax | 1.75 |
| 4. | White Petroleum Jelly | 3.25 |
| 5. | Glyceryl Monostearate | 3.50 |
| 6. | Stearic Acid | 1.00 |
| 7. | Tween ® 80 | 1.00 |
| 8. | Glycerin | 5.00 |
| 9. | Propylene Glycol | 5.00 |
| 10. | Purified Water | 71.50 |
| 11. | Perfume | q.s. |

Procedure:
i) Mix Vernonia cinerea extract, Liquid Light Paraffin, White Bees Wax, White Petroleum Jelly, Glyceryl Monostearate, Stearic Acid and heat upto 60-70° C.
ii) Mix Tween 80, Glycerin, Propylene Glycol and Purified Water and heat 60-70° C.
iii) Add the bulk of step (i) into bulk of step (ii) with constant stirring and allow to cool.
iv) Add perfume and mix.

### Example-11

### Vernonia Cream

| S. No. | Ingredient | % w/w |
|---|---|---|
| 1. | Vernonia tomentosa extract | 2.00 |
| 2. | Light liquid paraffin | 8.00 |
| 3. | White Bees Wax | 1.75 |
| 4. | White Petroleum Jelly | 3.25 |
| 5. | Tween ® 80 | 1.00 |
| 6. | Glycerin | 5.00 |
| 7. | Propylene Glycol | 5.00 |
| 8. | Sodium Carboxymethylcellulose | 74.00 |
| | (Sodium CMC (5% solution)) | |

Procedure:
i) Mix Vernonia tomentosa extract, Liquid Light Paraffin, White Bees Wax, White Petroleum Jelly and heat upto 60-70° C.
ii) Mix Tween ® 80, Glycerin, Propylene Glycol and Sodium CMC solution and heat 60-70° C.
iii) Add the bulk of step (i) into bulk of step (ii) with constant stirring and allow to cool to obtain the desired product.

### Example-12

### Cream

| S. No. | Ingredient | mg/gm |
|---|---|---|
| 9. | Vernonia latifolia extract | 1.00 |
| 10. | Aloe barbadensis extract | 0.50 |
| 11. | Propylene glycol | 50.00 |
| 12. | Titanium dioxide | 10.00 |
| 13. | Stearic acid | 130.00 |
| 14. | Cetyl alcohol | 10.00 |
| 15. | Isopropyl myristate | 60.00 |
| 16. | Sorbitan stearate | 20.00 |
| 17. | Methyl paraben | 1.50 |
| 18. | Propyl paraben | 0.30 |
| 19. | Corn oil | 50.00 |
| 20. | Glycerin | 50.00 |
| 21. | Sorbitol solution | 30.00 |
| 22. | Veegum HV | 10.00 |
| 23. | Sodium carboxymethylcellulose (Sodium CMC) | 3.00 |
| 24. | Tween ® 80 | 15.00 |
| 25. | Purified water | q.s. |

Procedure:
i) Vernonia latifolia extract, Aloe barbadensis extract, Methyl paraben and Propyl paraben are dispersed in Propylene glycol; the mixture heated to 55-60° C.; Titanium dioxide is added to it and stirred well.
ii) Stearic acid, Cetyl alcohol, Isopropyl myristate, Sorbitan stearate, and Corn oil are heated to 70°-75° C.
iii) In another vessel, Sorbitol solution and Tween ® 80 are taken.
iv) Veegum HV is separately hydrated in the Purified water.
v) Sodium carboxymethylcellulose (sodium CMC) is separately hydrated in Glycerin.
vi) The material of step (iv) and step (v) are added to the material of step (iii) and heated to 70°-75° C.
vii) The material of step (ii) and step (vi) are mixed and cooled.
viii) When the material of step (vii) attains a temperature of 50°-55° C., the material of step (i) is added to it.
ix) The mixture of step (vii) is allowed to cool to room temperature to obtain the cream.

### Example-13

### Cream

| S. No. | Ingredient | mg/gm |
|---|---|---|
| 1. | Vernonia anthelmentica extract | 0.50 |
| 2. | Serenoa repens extract | 0.30 |
| 3. | Propylene glycol | 50.00 |
| 4. | Titanium dioxide | 10.00 |
| 5. | Glyceryl monostearate | 90.00 |
| 6. | Hydrogenated lanolin | 30.00 |
| 7. | Corn oil | 40.00 |
| 8. | Simethicone | 1.50 |
| 9. | Span ® 60 | 20.00 |
| 10. | Hydroxyethyl cellulose | 20.00 |
| 11. | Glycerin | 50.00 |
| 12. | Sorbitol | 30.00 |
| 13. | Sodium carboxymethylcellulose (Sodium CMC) | 1.50 |
| 14. | Propyl paraben | 0.30 |
| 15. | Methyl paraben | 1.50 |
| 16. | Tween ® 80 | 15.00 |
| 17. | Purified water | q.s. |

Procedure:
i) Vernonia anthelmentica extract, Serenoa repens extract, Methyl paraben and Propyl paraben are dispersed in Propylene glycol; Titanium dioxide is added to it and stirred well.
ii) Glyceryl monostearate, Hydrogenated lanolin, Corn oil, Simethicane, and Span ® 60 are taken.
iii) In cool Purified water, Hydroxyethyl cellulose is dissolved; Sorbitol and Tween ® 80 is added to it and the mixture is heated to 70-75° C.
iv) Separately Sodium carboxymethylcellulose (sodium CMC) is dispersed in Glycerin and added to the material of step (iii).
v) The material of step (ii) is added to the material of step (iv) and allowed to cool with stirring.
vi) When a temperature of 50-55° C. is attained, the material of step (i) is added, stirred, and allowed to cool to room temperature to obtain the cream.

### Example-14

### Cream

| S. No. | Ingredient | Quantity (mg/g) |
|---|---|---|
| 1. | Vernonia anthelmentica extract | 0.01 |
| 2. | Soft paraffin | 350.00 |
| 3. | Liquid paraffin | 80.00 |
| 4. | Sorbitan monooleate | 50.00 |
| 5. | Citric acid | 1.00 |
| 6. | Sodium citrate | 2.00 |
| 7. | Purified water | q.s. to 1 g |

Procedure:
i) Soft paraffin, Liquid paraffin and Sorbitan monooleate are mixed at about 45° C. by continuous stirring to obtain a homogeneous dispersion.
ii) Vernonia anthelmentica extract is added to dispersion of step (i) with stirring.
iii) Citric acid and Sodium citrate are dissolved in a part of Purified water to make a solution.
iv) The material of step (iii) to step (ii) is added with continuous stirring at about 45° C. followed by the addition of the remaining part of Purified water and stirring.
v) The material of step (iv) is cooled to room temperature to obtain the desired product.

### Example-15

### Gel

| S. No. | Ingredient | Quantity (g/100 gm) |
|---|---|---|
| 1. | Vernonia sp. extract | 0.05 |
| 2. | Dimethylacetamide | 10.00 |
| 3. | Ethyl Alcohol | 20.00 |
| 4. | Acetone | 5.00 |
| 5. | Cremophor ® RH40 | 1.00 |
| 6. | Propylene glycol | 20.00 |
| 7. | Carbopol 934 | 1.20 |
| 8. | Purified water | 20.00 |
| 9. | Diethylamine | 0.60 |

Procedure:
i) Dimethylacetamide was mixed with Ethyl alcohol and Acetone in a container with stirring.
ii) To the mixture obtained, Vernonia sp. extract was added and stirred.
iii) Propylene glycol and Cremophor ® RH40 were dispersed in water, and were mixed in homogenizer. To the mixture obtained, Carbopol 934 was added and homogenized.
iv) The mixture obtained in step (ii) was added to the mixture obtained in step (iii) under stirring.
v) The mixture obtained was neutralised by slow addition of Diethylamine with slow stirring to produce the gel.

### Example-16

### Nebulizable Dispersion

Vernonia sp. extract 0.06 mg, Corticosteroid 0.012 mg and Propylene glycol 5.0 g are dispersed homogeneously in Purified water 10.0 g. The said dispersion is filled into a suitable container for atomization such as a nebulizer.

### Example-17

### Solution for Atomization

Vernonia sp. extract 0.05 mg and Minoxidil 0.05 mg are dispersed homogeneously in Ethanol-Purified water mixture (2.0 g & 6.0 g respectively); the said dispersion is filled into a suitable container.

### Example-18

### Ointment

| S. No. | Ingredient | Quantity (mg/g) |
|---|---|---|
| 1. | Vernonia anthelmentica extract | 0.05 |
| 2. | Ginkgo biloba extract | 0.05 |
| 3. | Lanoline | 10.00 |
| 4. | Eucalyptus oil | 0.40 |
| 5. | Peppermint oil | 0.10 |
| 6. | Liquid paraffin | q.s. to 1 g |

Procedure:
i) Eucalyptus oil and Peppermint oil is added to a part of Liquid paraffin and mixed with stirring.
ii) The material of step (i) and Lanoline are mixed at about 45° C. by continuous stirring to obtain a homogeneous dispersion.
iii) Vernonia anthelmentica extract and Ginkgo biloba extract are added to the dispersion of step (ii) with continuous stirring.
iv) The material of step (iii) is cooled to room temperature to obtain the desired product.

### Example-19

### Hair Oil

| S. No. | Ingredient | Quantity (% w/v) |
|---|---|---|
| 1. | Vernonia anthelmentica extract | 30.00 |
| 2. | Aloe barbadensis extract | 3.00 |
| 3. | Vegetable oil base | q.s. |

Procedure:
i) Vernonia ; sp. extract and Aloe barbadensis extract are prepared and mixed with the Vegetable oil base.
ii) The material of step (i) is filled into a suitable container.

### Example-20

### Shampoo

| S. No. | Ingredient | Quantity (% w/v) |
|---|---|---|
| 1. | Vernonia sp. extract | 20.00 |
| 2. | Ketoconazole | 2.00 |
| 3. | Shampoo Base | q.s. |

Procedure:
i) Vernonia sp. extract and ketoconazole are mixed together.
ii) The material of step (i) is incorporated into a Shampoo base.

### Example-21

### Tablet

| S. No. | Ingredient | mg/tablet |
|---|---|---|
| 1. | Vernonia sp. extract | 50.0 |
| 2. | Microcrystalline cellulose | 100.0 |
| 3. | Mannitol | 80.0 |
| 4. | Croscarmellose sodium | 10.0 |
| 5. | Lactose | 60.0 |
| 6. | Talc | 4.0 |
| 7. | Colloidal silicon dioxide | 10.0 |
| 8. | Croscarmellose sodium | 10.0 |

Procedure:
i) Vernonia sp. extract, Microcrystalline cellulose, Mannitol, Croscarmellose sodium and Lactose are sifted and mixed together.
ii) The material of step (i) is compacted.
iii) The compacts of step (ii) are passed through sieve and mixed.
iv) Talc, Colloidal silicon dioxide and Croscarmellose sodium are passed through fine sieve and mixed together.
v) The material of step (iii) is mixed with material of step (iv).
vi) The material of step (v) is compressed into tablets at an average weight of 400 mg ± 2%.
vii) The tablets are packed in air-tight packages.

### Example-22

### Capsule

| S. No. | Ingredient | mg/capsule |
|---|---|---|
| 1. | Vernonia sp. extract | 25.0 |
| 2. | Microcrystalline cellulose | 150.0 |
| 3. | Mannitol | 65.0 |
| 4. | Lactose | 50.0 |
| 5. | Talc | 3.0 |
| 6. | Sodium starch glycollate | 17.0 |
| 7. | Colloidal silicon dioxide | 15.0 |

Procedure:
i) Vernonia sp. extract, Microcrystalline cellulose, Lactose and Mannitol are sifted and mixed together.
ii) Talc, Sodium starch glycollate and Colloidal silicon dioxide are passed through fine sieves individually and then mixed together.
iii) The materials of step (i) and (ii) are mixed.
iv) The material of step (iii) is filled into empty hard gelatin capsules.

1. A novel composition for hair loss prevention and/or hair growth promotion comprising at least one agent(s) derived from a natural source or synthetic source or semi-synthetic source as the active agent, either alone or in combination with other active agent(s) and optionally one or more excipient(s).

2. A composition according to claim 1, comprising an extract obtained from the plant Vernonia sp. as active agent either alone or in combination with other active agent(s) and optionally with one or more excipient(s).

3. A composition according to claim 2, wherein the active agent for hair loss prevention and/or hair growth promotion is an extract obtained from the plant Vernonia noveboracense, Vernonia praealta, Vernonia tomentosa, Vernonia anthelmintica, Vernonia amygdalina, Vernonia cinerea, or mixtures thereof.

4. A composition according to claim 2, comprising an extract obtained from the plant Vernonia anthelmintica.

5. A composition according to claim 1, wherein the extract for hair loss prevention and/or hair growth promotion comprises of one or more components selected from a group comprising phytosterols, fatty acids or fatty acid esters, carotenoids, or mixtures thereof.

6. A composition according to claim 2, wherein the active agent comprises an extract obtained from the plant Vernonia sp. combined with at least one other extract obtained from a natural source selected from a group comprising Aloe (Aloe barbadensis), Burdock (Arctium minus), Capsicum (Capsicum annuum L), Ginger (Zingiber officinale), Ginkgo (Ginkgo biloba), Green Tea (Camellia sinesis), Hip (Rosa canina), Lavender (Lavendula officinale), Milfoil (Achillea millefolium), Nettles (Urtica dioica), Onion (Allium cepa), Pygeum (Pygeum africanum), Rattanjot (Arnebia sp.), Red Pepper (Capiscum annum), Rosemary (Rosamarinus officinalis), Safflower Oil (Carthamus tinctorious), Saw Palmetto (Serenoa repens), and Tea Tree Oil (Melaleuca alternifolia), or mixtures thereof.

7. A composition according to claim 2, wherein the active agent comprises an extract obtained from the plant Vernonia sp. combined with at least one other allopathic drug selected from 5α-DHT inhibitors, Super oxide dismutases mimetics, Vasodilators, Prostaglandin-H synthase-1 activators and Potassium channel openers.

8. A composition according to claim 2, wherein the active agent comprises an extract obtained from the plant Vernonia sp. combined with at least one other allopathic drug selected from a group comprising corticosteroids, dithranol, tretinoin, minoxidil, zinc, irritants, finasteride, skinoren/azelaic acid, cyproterone acetate with ethinyloestradiol, cimetidine, cyproterone acetate, spironolactone, ketoconazole, antidepressant, triamcinolone acetonide, hydroxychloroquine, penicillin, or mixtures thereof.

9. A composition according to claim 1, wherein the excipient(s) are selected from a group comprising diluents, disintegrants, binders, anti-adherants, glidants, lubricants, antioxidants, buffering agents, colorants, flavoring agents, coating agents, solvents, osmotic agents viscosifying agents, waxes, wetting agents, emulsifying agents, solubilizers, stabilizers, buffering agents, chelating agents, vehicles, preservatives, surfactants, deodorants, colorants, bulking agents, hydrophilic polymers, tonicity adjusting agents, local anesthetics, pH adjusting agents acids, sugar alcohol, reducing sugars and non-reducing sugars, or mixtures thereof.

10. A composition according to claim 1, which is in the form of a topical preparation selected form a group comprising liquid, cream, gel, lotion or spray.

11. A composition according to claim 1, wherein the composition is useful as a pharmaceutical product.

12. (canceled)

13. A process for preparation or extraction of the hair loss preventing and/or hair growth promoting agent from a natural source for use as an active agent according to claim 1 or 2, wherein said process comprises the following steps:
i) extraction of dried and powdered plant or part(s) of plant with a non-polar solvent or mixtures thereof,
ii) installation of the extract to remove the solvent,
iii) optionally, further extraction of the residue with a polar solvent or mixtures thereof, and
iv) optionally, distillation of the extract to remove the solvent to obtain the desired extract preferably as a powder.

14. A process for preparation or extraction of the hair loss preventing and/or hair growth promoting agent from a natural source for use as an active agent according to claim 1 or 2, wherein said process comprises the following steps:
i) extraction of dried and powdered plant or part(s) of plant with a polar solvent or mixture thereof,
ii) optionally, distillation/concentration of the extract to remove/reduce the solvent, and
iii) optionally drying the extract to remove the solvent to obtain the desired extract preferably as a powder.

15. A process for preparation or extraction of the hair loss preventing and/or hair growth promoting agent from a natural source for use as an active agent according to claim 1 or 2, wherein said process comprises the following steps:
i) expression of the juice of the fresh plant or part(s) of plant optionally with addition of a polar solvent or mixture thereof,
ii) filtration of the juice,
iii) optionally, distillation/concentration of the extract to remove/reduce the solvent, and
iv) optionally drying the extract to remove the solvent to obtain the desired extract preferably as a powder.

16. A process for preparation of a composition according to claim 1 or 2, which comprises the following steps:
i) mixing the hair loss preventing and/or hair growth promoting agent active agent(s) with one or more excipient(s), and
ii) formulating the mixture into a suitable dosage form.

17. A method of using a composition according to claim 1 or 2, which comprises administering to a subject in need thereof an effective amount of the composition.

18. A method of using a composition according to claim 1 or 2, which comprises administering to a subject in need thereof an effective amount of the composition for the management of one or more hair disorders selected from a group comprising alopecia greata, androgenetic alopecia, anagen effluvium (cancer treatment hair loss), self induced hair loss, telogen effluvium, scarring alopecia, syphilitic alopecia, scleroderma and tinea capitis.

19. (canceled)

20. (canceled)

21. A composition according to claim 1, wherein the composition is useful as a cosmetic product.

22. A composition according to claim 1, wherein the composition is useful as an ayuredic product.
